(19) United States
(12) Patent Application Publication
(10) Pub. No.: US 2005/0059152 A1
(43) Pub. Date: Mar. 17, 2005

(54) In vitro culture of mesenchymal stem cells (MSC) and a process for the preparation thereof for therapeutic use

(75) Inventors: Vivek Tanavde, Mumbai (IN); Prathibha Rai, Mumbai (IN); Khushnuma Soli Bharucha, Mumbai (IN)

Correspondence Address: LACKENBACH SIEGEL, LLP, LACKENBACH SIEGEL BUILDING, 1 CHASE ROAD, SCARSDALE, NY, 10583, US

(73) Assignee: Reliance Life Sciences Pvt. Ltd., Mumbai (IN)

(21) Appl. No.: 10/853,077

(22) Filed: May 25, 2004

(30) Foreign Application Priority Data
May 26, 2003 (IN) 532/MUM/2003

(51) Int. Cl. C12N 5/08

(52) U.S. Cl. 435/372

(57) ABSTRACT

Method of culturing mesenchymal stem cells (hMSC) by culturing the stem cells in a culture medium containing Cord Blood Serum, and a process for preparation thereof for therapeutic use.

### RELATED APPLICATION

This application claims priority to the Provisional Application No. 532/MUM/2003 filed on 26 May 2003.

### TECHNICAL FIELD

The present invention provides a method for culturing mesenchymal stem cells using cord blood serum, for therapeutic purposes in regenerative medicine. In particular the present invention provides the use of these cells in the treatment of cardiac disorders.

### BACKGROUND OF THE INVENTION

Stem cells are special cells that have the ability to develop into many different types of tissue: bone, muscle, nerve, etc. Stem cells are separated into three (3) distinct categories viz. Totipotent, Pluripotent, and Multipotent.

These cells are found in the bone marrow, blood, dermis, and periosteum. They are capable of differentiating into any of the specific types of mesenchymal or connective tissues like cartilage, bone, muscle, fat and tendon depending upon various influences from bioactive factors, such as cytokines. A population of these stem cells has been shown to be present in the embryonic limb buds of chicken, mouse and human. Such cells have also been shown to exist in post-natal and adult organisms. Friedenstein, Owen and other researchers reported that cells derived from bone marrow have the capability to differentiate into osteogenic cells when assayed in diffusion chambers. It has been documented that such cells when loaded in ceramic cubes and implanted to subcutaneous or intramuscular sites, possess the ability to form bone and cartilage tissue. Furthermore the periosteum has been reported to contain mesenchymal stem cells with the same ability to form bone or cartilage in ceramic cubes or diffusion chambers.

Mesenchymal Stem Cells are pluripotent cells that can be isolated from adult bone marrow and can be induced in vitro and in vivo to differentiate into a variety of mesenchymal tissues, including bone, cartilage, tendon, fat, bone marrow stroma, and muscle. Human umbilical cord blood is also a good source of mesenchymal stem cells. However the yield of mesenchymal cells from cord blood is much lower as compared to bone marrow. Therefore even though cord blood units are being banked in large numbers for conventional hematopoietic stem cell transplants, its potential as a source of mesenchymal stem cells is largely unexplored. The present invention aims to address this issue by developing a protocol that could be used for the rapid expansion of cord blood mesenchymal stem cells. Such expansion of cord blood derived mesenchymal stem cells would make them therapeutically useful.

Despite advances in the treatment of myocardial infarction (MI), congestive heart failure secondary to infarction continues to be a major complication. The cardiomyoctyes lost during an MI cannot be regenerated, and the extent of the loss is inversely related to cardiac output, pressure- generating capacity, and ultimately survival.

Cell therapy, or the supplementation of tissue with exogenous cells, has previously been used in the treatment of disease in which terminally differentiated cells are irreparably damaged. Recently, it has been suggested that cell therapy with myoblasts may be effective in the treatment of MI. Cell therapy has been used effectively in the treatment of a variety of human disorders from Parkinson's disease to diabetes, and holds promise in the therapy of many diseases in which non-regenerative cell death or abnormal cellular function plays a role.

Mesnchymal Stem cells possess the ability to differentiate into a variety of Mesenchymal tissues including bone, cartilage, muscle, fat, dermis as demonstrated in a number of organisms including humans (Bruder, J Cellul Biochem 1994). Recent studies indicate that bone marrow stem cells can differentiate into nonhematopoietic cells of ectodermal, mesodermal, and endodermal tissues other than hematopoietic tissues, including liver, pancreas, kidney, lung, skin, gastrointestinal tract, heart, skeletal muscles, and neural tissues. Studies reporting the multipotentiality of BM cells have become a focus of interest because they suggest that clinical applications could be at hand using easily obtainable cells in the treatment of tissue damage or degenerative diseases. (Heike Int J Hematol 2004). Adult MSCs are also easy to manipulate in vitro. It is these properties of adult MSCs that have made them the focus of cell-mediated gene therapy for skeletal tissue regeneration. Adult MSCs engineered to express various factors not only deliver them in vivo, but also respond to these factors and differentiate into skeletal specialized cells. This allows them to actively participate in the tissue regeneration process. (Gazit Gene Ther. 2004).

The inventors of the present invention have discovered the optimal seeding density of mesenchymal stem cells to obtain good expansion of these cells in in vitro culture. This observation will be useful in the development of a large scale culture system for rapid expansion of mesenchymal stem cells in a clinical setting. Such large scale cultures can utilize but are not limited to vessels like cell culture bags or small scale bioreactors.

Mesenchymal stem cells isolated by the present invention may be used to reconstruct damaged myocardium in patients who have undergone multiple myocardial infarcts, which has resulted in loss of cardiac muscles. Such a heart is not fully functional and the condition is potentially life threatening as the cardiomyocytes lost during infarction cannot be regenerated and the extent of loss is inversely related to cardiac output and ultimately survival. Cultured Mesenchymal stem cells described in the present invention may be surgically implanted into the damaged myocardium, where they may grow and transform themselves into heart muscles. This procedure may improve the function of the heart.

The present invention is not limited to culturing of human mesenchymal stem cells derived from bone marrow and cord blood, it also includes culturing of animal mesenchymal stem cells.

Conventionally, patients have to be transplanted with a healthy heart from a donor who is tissue matched. Unfortunately, such donors are not easily available and the procedure is not very successful in improving the patient's life. However, the present procedure suggests that cultured mesenchymal stem cells may be beneficial for cellular transplantation therapy of myocardial infarction in humans.

Current therapeutic modalities for the treatment of end stage cardiac failure are limited and include medical therapy, mechanical left ventricular assist devices and cardiac transplantation. Cardiac transplantation is the treatment of choice for end stage cardiac disease, but is hampered by the limited availability of donor organs, the complications of immunosuppressive therapy and the long term failure of grafted organs. Thus the development of alternative therapeutic strategies to combat intractable cardiac disease remains an important therapeutic goal. Augmentation of myocardial performance in experimental models of heart failure and infarction has been achieved by transplantation of exogenous cells into the damaged myocardium.

In light of the foregoing, is a need to develop a method which obviates the complications associated with the surgical therapies. Strategies are being pursued to develop effective therapy for treating cardiac disorders. Cell implantation offers hope for actually replacing cells that has been damaged through cardiac disease or degeneration to regenerate or repair cardiac muscles. Researchers have used mesenchymal stem cells as a new type to engineer heart valves in the laboratory. The cells are seeded on heart valve scaffolds made from bioabsorbable materials and grown in a pulse duplicator system, called leaflets or cusps.

Mesenchymal stem cells have been used to regenerate or repair cardiac muscle that has been damaged through disease or degeneration. Cardiac muscle does not normally have reparative potential. The mesenchymal stem cells differentiate into cardiac muscle cells and integrate with the healthy tissue of the recipient to replace the function of the dead or damaged cell, thereby regenerating the cardiac muscle as a whole. Rat & mouse clonal embryonic cell lines have been shown to transform into myoblasts and form myotubes after exposure to 5-azacytidine or 5-azadeoxycytidine. The same cells also exhibit adipogenic and chondrogenic phenotypes. The myogenic potential of mesenchymal stem cells has been well established.

A cardiomyogenic cell line has been established from murine bone marrow mesenchymal stem cells (Makino J Clin Invest 1999). After prolonged treatment with 5-azacytidine, these cardiomyogenic cells formed myotubes connected by intercalated discs, which beat synchronously. A study by Tomita (Circulation 1999) has shown that bone marrow mesenchymal stem cells are capable of stable cardiac engraftment and site specific differentiation in myocardial scar tissue in the rat cryoinjury model of infarction. Similarly, scientists have confirmed that the cardiac environment is permissive for cardiomyocyte differentiation of bone marrow mesenchymal stem cells even in the absence of 5-azacytidine pretreatment. In a study by Wang (J Thorac Cardiovasc Surg 2000), bone marrow mesenchymal stem cells adopted a differentiated cardiomyocyte phenotype and aligned and formed intercalated discs with host cardiomyocytes when directly implanted into the non-injured rat heart. Based on these observations, the authors of the present invention have devised methods for the culture of mesenchymal stem cells from bone marrow and sought to assess their role in cardiac repair.

Although mesenchymal stem cells are present at very low frequencies in bone marrow, inventors of the present invention have discovered a method by which these cells can be amplified in vitro to larger quantities in cell culture medium. The present invention provides the use of sera separated from umbilical cord blood for growing mesenchymal stem cells for therapeutic purposes in regenerative medicine.

Cord blood serum (CBS) is a rich source of different cytokines required for growth and survival of different types of stem cells like hematopoietic stem cells. Lam & co-workers (Transfusion 2001) have used autologous cord blood plasma for the expansion of cord blood hematopoietic stem cells. Cord blood plasma has also been used for the selective expansion of lymphocytes (U.S. Pat. No. 6,610,542, U.S. Pat. No. 6,194,207). Moreover, the placenta is the site of major endocrine activity, including synthesis of a broad range of steroid and peptide hormones, growth factors, cytokines, and other bioactive factors. Some of these growth factors like IGF, TGF-β, VEGF are produced by the placental trophoblast. Since there is an exchange of nutrients between the placental and cord blood, it is likely that a lot of these growth factors maybe present in cord blood also. Broxmeyer et al (Blood Cells 1994) have assayed for a number of such co-stimulating factors present in cord blood plasma. Fetal plasma has also been shown to stimulate the activation of endothelial cells (Wang, Am J Obstet Gynecol 2003). Since it is established that primitive hematopoietic stem cells are present in human umbilical cord blood, it is possible that cord blood serum may be important in maintaining the ‘stemness’ of these cells.

The inventors of the present invention have devised a method for culturing MSC using human CBS. Till date there are no publications or patents on CBS being used for culturing MSC. Also, it is likely that cord blood serum may maintain the ‘stemness’ of other adult stem cells like mesenchymal stem cells. The present invention utilizes this property of cord blood serum to grow mesenchymal stem cells from bone marrow and as well as cord blood.

Until the present, mesenchymal stem cells are being cultured in animal serum such as fetal bovine serum (FBS), human adult blood serum or a complex mixture of growth factors derived from recombinant methods. However, these conventional culture media are associated with shortcomings and risks. Stem cells from adult/fetal as well as other sources are being widely used to regenerate tissues in patients after they have degenerated. For this purpose, these cells have to be grown in tissue culture for varying periods of time using defined media, the principle constituent of which is animal serum such as Fetal Bovine Serum (FBS).

FBS is the most widely used serum in the culturing of cells, tissues and organs in vitro, in industry, medicine, and science. FBS has been shown to be essential for adhesion, proliferation and differentiation of the cells. FBS has also been used extensively for the culture of Mesenchymal Stem Cells (U.S. Pat. No. 6,355,239, U.S. Pat. No. 6,368,636). However, animal serum such as FBS can be infected with several pathogens such as prions. Moreover, FBS being of animal origin is unsuitable for infusion into human beings. Trace amounts of FBS are retained on cell surfaces even after extensive washing and could lead to toxicity in humans. To overcome this limitation, attempts have been made to culture bone marrow stromal cells by substituting FBS with human AB serum (Yamaguchi M Transfusion 2002). However, adult human serum lacks many of the growth factors needed for survival and expansion of stem cells. Hence, it is not widely used for culturing of cells, tissues and organs in vitro.

Several investigators have also tried to use a complex mixture of recombinant growth factors for cell culture which are known to influence growth and differentiation of stem cells. However, the success is limited and the added cost of using these growth factors makes the clinical use of these cultured stem cells, a very expensive affair. Hence, it is impractical to device a completely defined medium comprising of recombinant growth factors for the growth of MSC for therapeutic use.

There is a dire need to find an adequate substitute for conventional culture media for growing Human Mesenchymal Stem Cells. Looking to the need of the hour, the present inventors have resolved the above issue of concern and have come out with a solution which will be of utmost importance in the field of regenerative medicine. The inventors have come out with a unique component for culturing Human Mesenchymal Stem Cells.

The novelty of the present invention lies in the use of human CBS for culturing MSC and replacing FBS by CBS.

Since ontogenically human cord blood serum (CBS) is closer to FBS, the inventors of the present invention decided to replace FBS by human cord blood serum in the invention. Also CBS, like FBS is found to be rich in growth factors. Taking this factor in mind, the inventors of the present invention, have investigated a method of growing Human Mesenchymal Stem Cells in cord blood serum.

The present invention solves the issue of infusion of xenogenic sera into humans with a unique solution that will be of utmost importance in the field of regenerative medicine. The present invention is advantageous over the prior art as it obviates the problems associated with conventional culture media for growing human mesenchymal stem cells. The present invention has solved the problems by culturing human stem cells in umbilical cord blood serum.

### OBJECTS OF THE INVENTION

It is an object of the present invention to culture mesenchymal stem cells for therapeutic purposes in regenerative medicine, in particular cardiac disorders.

It is also an object of the present invention to develop a method for culture of mesenchymal stem cells using cord blood serum for therapeutic purposes in regenerative medicine

It is still an object of the present invention to replace FBS the conventionally used serum for culturing MSC by CBS.

It is further object of the present invention to culture mesenchymal stem cells using a mixture of cord blood serum and a culture medium comprising of Dulbecco's modified Eagle's medium (DMEM) and Ham's F-12 medium.

It is still further object of the present invention to develop a method by which these cells can be amplified in vitro to larger quantities in the culture medium.

It is still further an object of the present invention to develop a method for culturing MSC using CBS for therapeutic purposes not limited to cardiac disorders, but also may be used in repair of bone, cartilage and neural disorders.

### SUMMARY OF THE INVENTION

In accordance with one aspect of this invention, there is provided the use umbilical cord blood sera for growing human stem cells and adult cells, like Human Mesenchymal Stem Cells (hMSC) for therapeutic purposes in regenerative medicine.

The inventors of the present invention have conducted research on human umbilical cord blood and have replaced conventionally used Fetal Bovine Serum (FBS) by cord blood serum for culturing MSC derived from bone marrow as well as umbilical cord blood.

As stated, human umbilical cord blood is a fetal product and a waste product during childbirth. During the gestation of the child in the mother's womb, the placenta and the blood present in the placenta nourish the developing fetus and are therefore rich in several growth promoting factors. The inventors of the present invention have taken advantage of this property of Human umbilical cord blood serum and have substituted it for FBS. Also, the umbilical cord blood serum being of human origin, the risk of transmission of virus and bacteria in using FBS for culturing MSC is eliminated by the present invention.

### DETAILED DESCRIPTION OF THE INVENTION

The following terms are used throughout the specification to describe the present invention.

The term “umbilical cord blood” or “cord blood” is used throughout the specification to refer to blood obtained from a neonate or fetus, most preferably a neonate and preferably refers to blood which is obtained from the umbilical cord or placenta of newborns. The use of cord or placental blood as a source of mononuclear cells is advantageous because it can be obtained relatively easily and without trauma to the donor. Cord blood cells can be used for auologous or allogenic transplantation when and if needed. Cord blood is preferably obtained by direct drainage from the umbilical vein.

The term “cell medium” or “cell media” is used to describe a cellular growth medium in which mononuclear cells and/or neural cells are grown. Cellular media are well known in the art and comprise at least of minimum essential medium plus optional agents such as growth factors, glucose, non-essential amino acids, insulin, transferrin and other agents well known in the art.

The term “non adherent cells” is used to describe cells remaining in suspension in the tissue culture flask at the end of the culture period. The term “adherent cells” is used to describe cells that are attached to the tissue culture plastic, and are detached from the flask by addition of enzyme free cell dissociation buffer from Gibco-BRL or by addition of trypsin-EDTA.

The term “mononuclear cells” is used to describe cells containing a single nucleus isolated from bone marrow or UCB using a density gradient of Ficoll™ or Percoll™ Mononuclear cells are obtained from bone marrow or umbilical cord blood and are used as a source of Mesenchymal Stem Cells.

In one aspect of the present invention, mononuclear cells obtained from the cord or bone marrow are grown in Mesenchymal Stem Cell proliferation medium i.e. a medium which efficiently grows MSC, not limited to at least a minimum essential medium supplemented with non-essential amino acids, glutamine and a serum of human or animal origin. A particularly preferred MSC proliferation medium is a medium which contains DMEM/F12 1:1 cell medium supplemented with glutamine 2 mM, sodium bi carbonate 3 mM and β-FGF 1-50 ng/ml and 1-50% CBS preferably between 2-30% of CBS. One of ordinary skill will readily recognize that any number of cellular media may be used to grow MSC from mononuclear cell fractions of umbilical cord blood or bone marrow and differentiate them in appropriate differentiation media.

The inventors have provided comparative data using CBS, FBS and commercially available MSC growth medium. The MSC in the present invention is isolated from human bone marrow, human umbilical cord blood, swine bone marrow.

Preparation of Umbilical Cord Blood Serum

Umbilical cord blood serum is prepared in the following manner:

The cord blood that would normally be discarded, is collected after delivery from pre-screened mothers, for infectious disease causing organisms, such as HIV 1 & 2, Hbs and HCV and sexually transmitted diseases. The cord blood is stored only with the mother's signed consent, and no collection is made if there are any complications during delivery.

The cord blood collection is made after the baby is separated from the clamped cord breaking the link between the baby and the placenta, therefore there is no harm caused to the baby. Trained staff drain the blood from the umbilical cord and placenta. Methods vary somewhat at different hospitals. Blood is collected from umbilical vein using the conventional blood bag containing no anticoagulants. The needle of the bag is inserted in to the vein and blood is allowed to flow into the blood bag. A good collection can exceed 100 ml. This blood is now allowed to clot at room temperature and transported to the processing area, which is a cGMP clean room. The clotting process is allowed to take place from 8-16 hours. The blood is then centrifuged at 1000 g in a blood bag centrifuge and the clear serum is collected into sterile containers. Serum from different donors is pooled to eliminate batch variations due to donor characteristics. The serum is now tested for sterility by microbiological assays for aerobic or anaerobic micro organisms. The complement is inactivated by keeping sera at 56° C. for ½ hour. Serum is aliquoted into 10 ml sterile vials and capped. Lot number and Batch number are fixed on it.

Preparation of Mononuclear Cells:

Mononuclear cells are isolated from bone marrow obtained from a resected swine rib or iliac aspirate from normal human volunteers in a conventional manner. When swine rib is used, it is opened laterally and the marrow is scraped from the inside of the rib. The marrow is then passed through a 20 G syringe followed by 23 G needle to break clumps. The marrow is then passed through a 70 mm nylon mesh to get rid of tissue debris and bone chips. These cells are then centrifuged on Percoll density gradient (density 1.073 gm/ml). When iliac crest aspirates are used, the diluted cell suspension is directly loaded onto a Percoll gradient. The cells are centrifuged at 400 G for 20 minutes and the ring formed at the interface is harvested, washed and plated for isolation of mononuclear cells. Enrichment of mononuclear cells is well known to the practitioners of the art. Umbilical cord blood from full term deliveries is collected and depleted of red blood cells using 3% dextran v/v as described in (Tanavde Ind J Med Res 1997). Briefly, red blood cell depletion is carried out using 3% v/v Dextran (high molecular weight) in the ratio of 1:1 with respect to the volume of blood. Leucocyte rich plasma is collected carefully and centrifuged. Cells are washed and layered on Percoll or Histopaque™ 1077. The tubes containing the cells layered onto the density gradient are centrifuged at 400 g for 30 minutes. Mononuclear cells are separated from the interface using pipettes. These cells are then washed, counted and suspended in MSC proliferation medium as described in this specification.

Characterization of Mononuclear Cells:

To determine the percentage of MSC in mononuclear cell fraction the following procedure is performed.

The mononuclear cells are also stained with fluorochrome conjugated antibodies for flow cytometry. Specified numbers of cells are taken in polystyrene round bottom tubes. These cells are then stained with anti-CD73-FITC, anti-CD73-PE, anti CD105-PE and anti-CD45-PerCP antibodies. The stained cells are then acquired and analyzed on a FACSCalibur flow cytometer. The same procedure is used for characterization of cultured MSC.

In accordance with a second aspect of this invention, the inventors of the present invention have come out with the invention of growing Mesenchymal Stem Cells (MSC) using cord blood serum and a method for preparation thereof for therapeutic purposes.

The present invention relates to a method of isolation and culture of mesenchymal stem cells comprising the following:
a) The mononuclear cell suspension is plated into tissue culture flasks so that mesenchymal stem cells can adhere for 24-72 hrs.
b) Transferring the resulting supernatant to fresh flasks for the remaining mesenchymal stem cells to adhere. These cultures are incubated at 37° C. in a 5% CO₂ in air incubator;
c) Cultures are fed every 3-5 days using the medium described above. Cultures reach confluence by 1.5-2.5 weeks. At this point they are transferred to fresh flasks and passaged for passages until sufficient cell numbers for transplantation are obtained.

As used herein the term “confluent” indicates that the cells have formed coherent cellular monolayer on the surface so that virtually all available surface is used leading to inhibition of cell proliferation.

In the present invention growth factors is selected from the group consisting of Epidermal growth factor (EGF), Nerve growth factor (NGF), Fibroblast Growth Factor (FGF), Transforming growth factor-β. (TGF-β) either singly or in a combination thereof.

In the present invention culture media is selected from the group consisting of Dulbecco's modified Eagle's medium, (DMEM), Hams F-12 medium, Iscoves modified Dulbeccos medium (IMDM), Roswell Park Memorial Institute medium (RPMI)

The marrow or isolated MSC can be autologous, allogeneic or from xenogeneic sources, and can be embryonic or from post-natal sources. Bone marrow cells may be obtained from iliac crest, femora, tibiae, spine, rib or other medullary spaces. Other sources of MSC include Fat, Periosteum, Skin, Skeletal muscle, Liver, Placenta, Blood and Umbilical Cord.

In conclusion the present invention provides an easy, simple, effective and economical method to amplify mesenchymal stem cells for therapeutic purpose by culturing them in cord blood serum.

The following examples are intended to illustrate the invention but do not limit the scope thereof.

### EXAMPLES

### Example 1

Effect of Cord Blood Serum on the Proliferation of Human Bone Marrow MSC:

Mononuclear cells from bone marrow were plated in Nunc T75 culture flasks in MSC proliferation medium containing DMEM/F12 (1:1) supplemented with FBS or CBS. The cells were seeded at a density of 1×10⁶ to 1×10⁷ cells/ml. After a fixed culture period of 1 week, the adherent cells were harvested, counted and analyzed for the expression of CD73, CD105 and CD45 markers. FIG. 1 shows the morphology of these cells cultured in the presence of FBS & CBS. These cells showed a fibroblast like morphology, which is typical of MSC. FIG. 2 shows the phenotype of these cells cultured in the presence of FBS v/s CBS. No significant difference was observed in the morphology & phenotype of these cells cultured in the presence of CBS or FBS. FIG. 3 shows the growth kinetics of cells cultured in the presence of FBS v/s CBS. Cells cultured in the presence of CBS showed a higher cell count as compared to those cultured in the presence of FBS. However after the third passage, cells cultured in the presence of CBS sloughed off the flask, whereas cells cultured in FBS containing medium continued to grow as an adherent monolayer. This is reflected in FIG. 3 where the cell count in the CBS flask has decreased at passage 4 as compared to passage 3.

### Example 2

Effect of Cord Blood Serum on the Proliferation of Swine MSC:

Mononuclear cells from swine rib bone marrow were plated in Nunc T75 culture flasks in MSC proliferation medium containing DMEM/F12 (1:1) supplemented with FBS or CBS. The cells were seeded at a density of 1×10⁶ to 1×10⁷ cells/ml. The MSC started to adhere and after about 7 days started to form colonies. Once the flask was full with these colonies, the adherent cells were harvested, counted and analyzed for the expression of CD45 marker. FIG.-4 shows the morphology of the adherent MSC cultured in FBS and CBS. A fibroblast like morphology typical of MSC is observed here also. FIG. 5 shows the phenotype of these cells cultured in the presence of FBS v/s CBS. No significant difference was observed in the phenotype of these cells cultured in the presence of FBS or CBS. Cells cultured in the presence of FBS or CBS were negative for CD45 marker. FIG. 6 shows the growth kinetics of cells cultured in the presence of FBS v/s CBS. Cells cultured in the presence of FBS or CBS showed no significant difference in the cell counts at the end of 3 passages.

### Example 3

Effect of Serum Free Medium on the Proliferation of MSC:

Mononuclear cells from human bone marrow were cultured in cell culture cassettes in commercially available serum free medium. Cells cultured in Nunc T75 culture flasks in MSC proliferation medium containing DMEM/F12 (1:1) supplemented with FBS served as controls. The cells were seeded at a density of 1×10⁶ to 1×10⁷ cells/ml. After about a week the MSC started to adhere and form colonies. After about 10-15 days, the adherent cells were harvested, counted and analyzed for the expression of CD73, CD105 and CD45 markers. FIG. 7 shows the morphology of MSC cultured in the presence of serum free medium and FBS. Morphologically the cells did not show any difference whether cultured in serum free medium or FBS. FIG. 8 shows the phenotype of these cells cultured in the presence of FBS v/s serum free medium. No significant difference was observed in the phenotype of these cells cultured in the presence of serum free medium or FBS. All cells were negative for CD45 marker and positive for CD73 and CD105 markers. FIG. 9 shows the growth kinetics of cells cultured in the presence of FBS v/s serum free medium. Cell counts were not significantly different of cells cultured in FBS or serum free medium up to 4 passages. Cells cultured in the presence of serum free medium could be cultured up to 5 passages with an increase in their numbers.

### Example 4

Effect of Cord Blood Serum on the Proliferation of Cord Blood MSC:

Mononuclear cells from cord blood were plated in Nunc T75 culture flasks in MSC proliferation medium containing DMEM/F12 (1:1) supplemented with 10% FBS or CBS. The cells were seeded at a density of 10×10⁶ to 20×10⁶ cells/ml. After 1 week of culture the cells started adhering to the cell surface. The adherent cells were allowed to grow till the time they became confluent. The cells were then harvested, counted and analyzed for the expression of CD73, CD105 and CD45 markers. FIG. 10 shows the morphology of these cells cultured in the presence of FBS & CBS. These cells showed a fibroblast like morphology, which is typical of MSC. After the first passage, these cells showed a CD45-/CD73+/CD105+ phenotype, which is characteristic of MSC. This phenotype was maintained for up to 6 passages. We were not able to follow these cultures beyond 6 passages. No significant difference was observed in the phenotype of these cells cultured in the presence of CBS or FBS. FIG. 11 shows the phenotype of these cells cultured in the presence of FBS v/s CBS from a representative experiment. FIG. 12 shows the growth kinetics of cells cultured in the presence of FBS v/s CBS. As observed from this figure, no significant difference in cell numbers was observed in cells cultured in the presence of FBS v/s CBS in the first 2 passages. However at the 3^{rd} passage, cells cultured in CBS showed a 3 fold higher cell count as compared to cells cultured in FBS.

Therefore taken together the above data suggests that no difference in morphology & phenotype was observed in MSC cultured in the presence of FBS v/s CBS. Since CBS is capable of supporting the growth of MSC from CB, it can be used as a replacement for FBS in these cultures.

### BRIEF DESCRIPTION OF DRAWINGS

The following figures, which are in the form of photographs are part of the present specification and are incorporated to further demonstrate certain aspects of the present invention. The invention may be better understood by reference to these figures in combination with the detailed description presented herein.

FIG. 1: This Figure shows the morphology of human bone marrow MSC cultured in the presence of FBS & CBS. Fibroblast like spindle shaped cells are seen after about 7 days of culture in both these cultures.

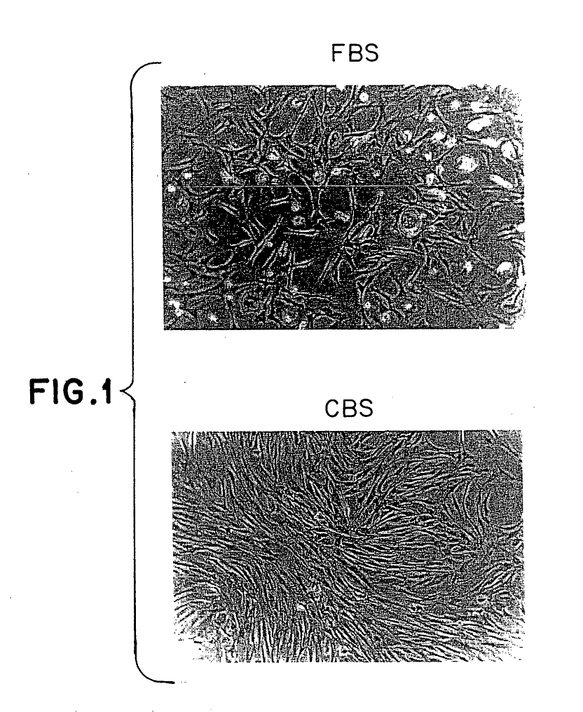

FIG. 2: This Figure shows the Flow Cytometric characterization of human MSC cultured in the presence of FBS & CBS. Harvested cells were stained with CD45, CD73 and CD105 markers. Cells cultured in medium containing FBS or CBS were negative for CD45 marker and positive for CD73 and CD 105.

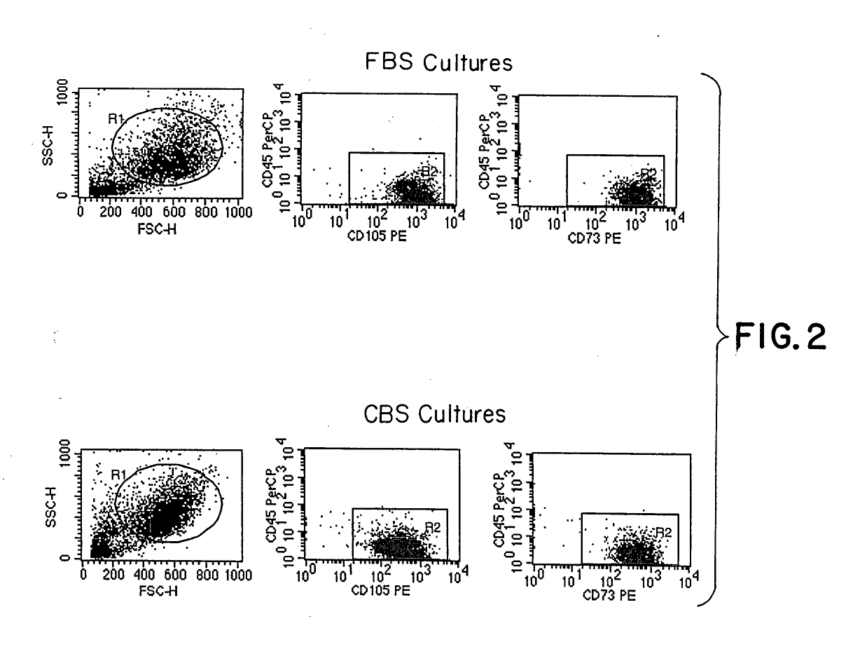

FIG. 3: This Figure shows the Growth Kinetics of human MSC cultured in presence of medium containing FBS & CBS. Cells cultured in the presence of CBS showed a higher cell count as compared to those cultured in the presence of FBS. Cell count in CBS containing medium decreased after the third passage as compared to cells cultured in FBS containing medium.

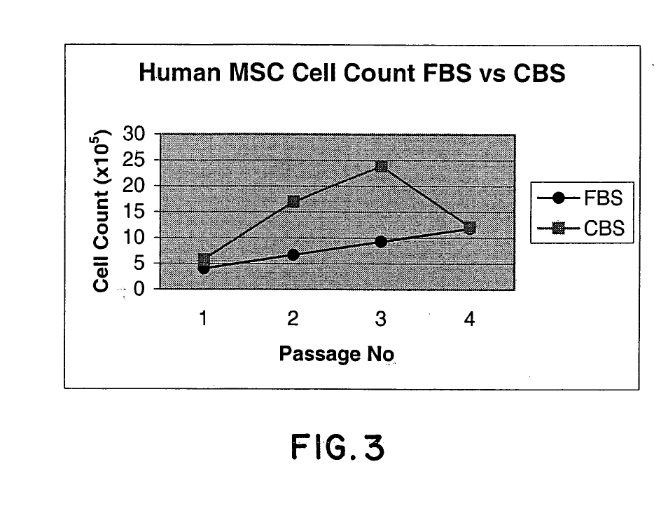

FIG. 4: This Figure shows the Morphology of the adherent swine MSC cultured in FBS and CBS. Cells showed a typical fibroblast like morphology.

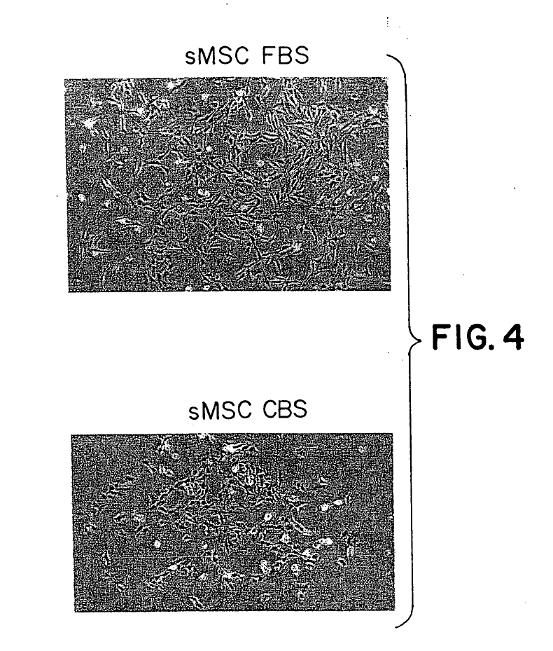

FIG. 5: This Figure shows the Flow Cytometric characterization of swine MSC cultured in FBS containing medium and CBS containing medium. Cells were labeled with CD45 marker, and were found to be CD45 negative.

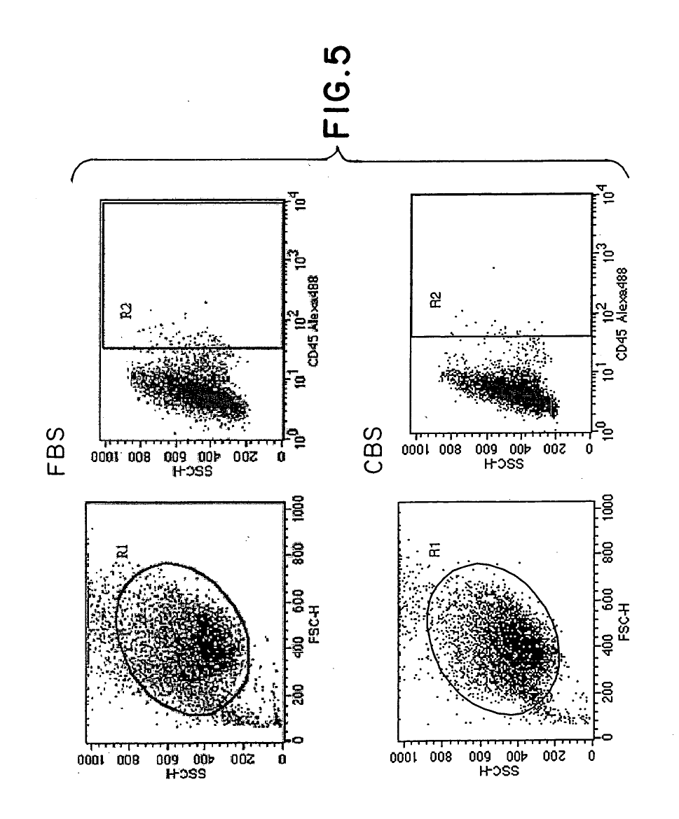

FIG. 6: This Figure shows the Growth Kinetics of swine MSC cultured in the presence FBS and CBS. No significant difference was observed in the cell counts of cells cultured in presence of FBS or CBS.

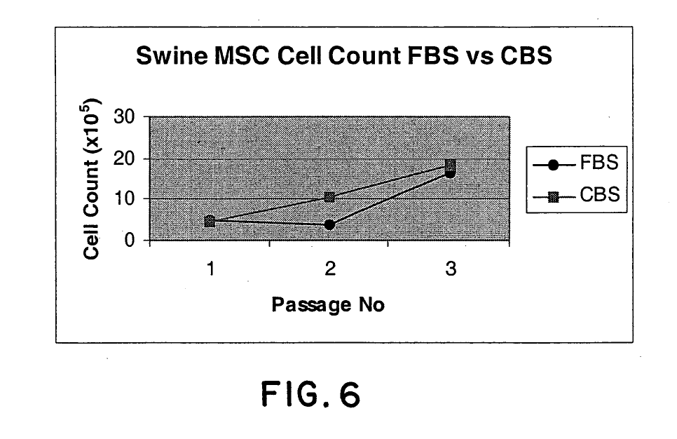

FIG. 7: This Figure shows the Morphology of human MSC cultured in the presence of serum free medium and FBS containing medium. Adherent cells cultured in the presence of serum free medium and FBS containing medium showed a fibroblast like morphology.

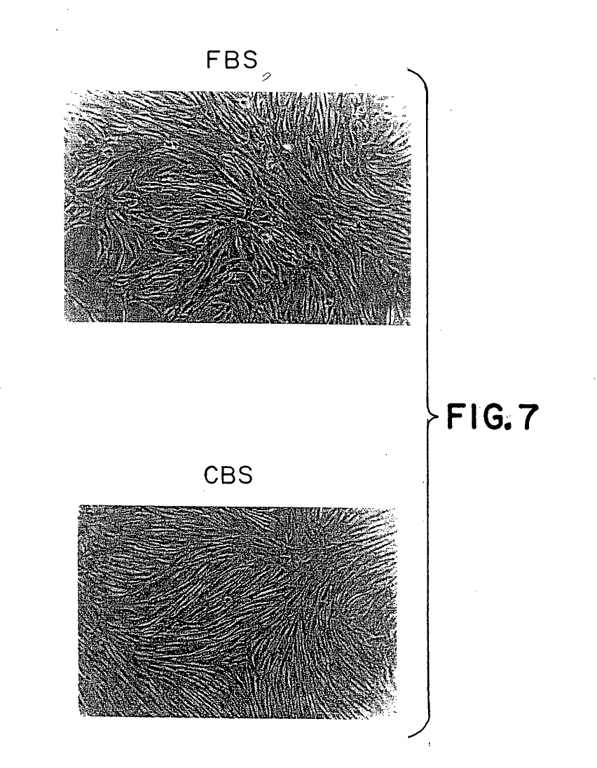

FIG. 8: This Figure shows the Flow Cytometric characterization of human MSC cultured in presence of serum free medium and FBS containing medium. Cells were labeled with CD45, CD73 and CD105 markers. All cells were found to be negative for CD45 and positive for CD73 and CD 105 markers.

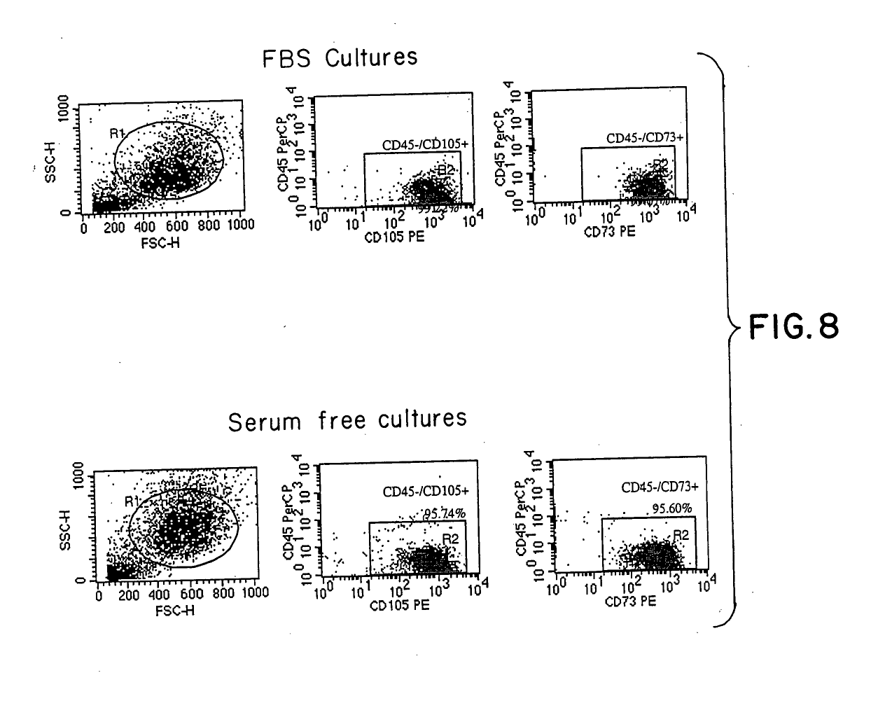

FIG. 9: This Figure shows the Growth Kinetics of human MSC cultured in presence of serum free medium and FBS containing medium. Cell counts were not significantly different of cells cultured in FBS or serum free medium up to 4 passages. Cells cultured in the presence of serum free medium could be cultured up to 5 passages with an increase in their numbers.

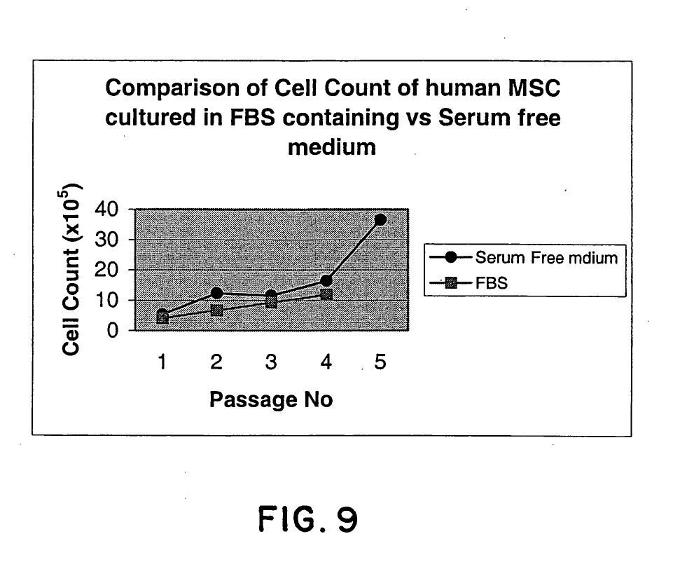

FIG. 10: This Figure shows the Morphology of cord blood MSC cultured in the presence of medium containing FBS & medium containing CBS. Cells showed a fibroblasts like spindle shaped morphology.

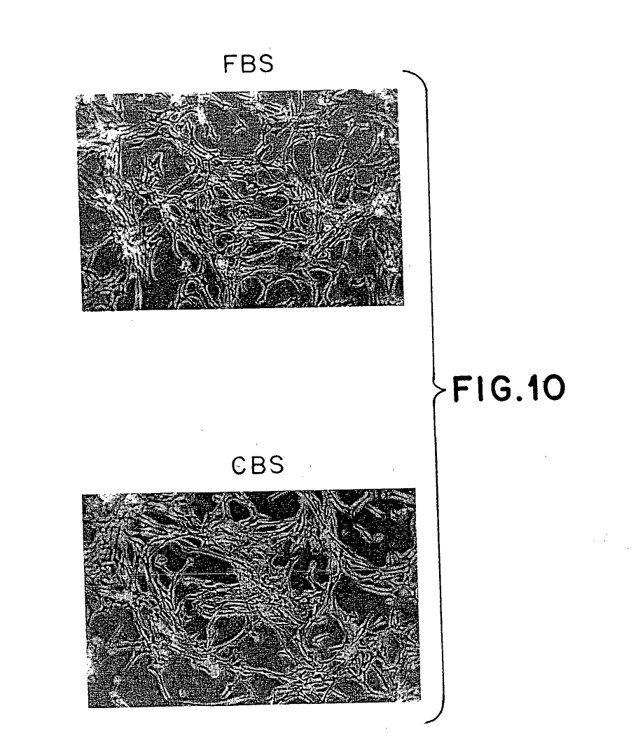

FIG. 11: This Figure shows the Flow Cytometric characterization of cord blood MSC cultured in the presence of medium containing FBS and CBS. The adherent cells from these cultures were labeled with CD45, CD73 and CD 105 antibodies. After Passage 1, all cells were found to be negative for CD45 and positive for CD73 and CD 105 markers.

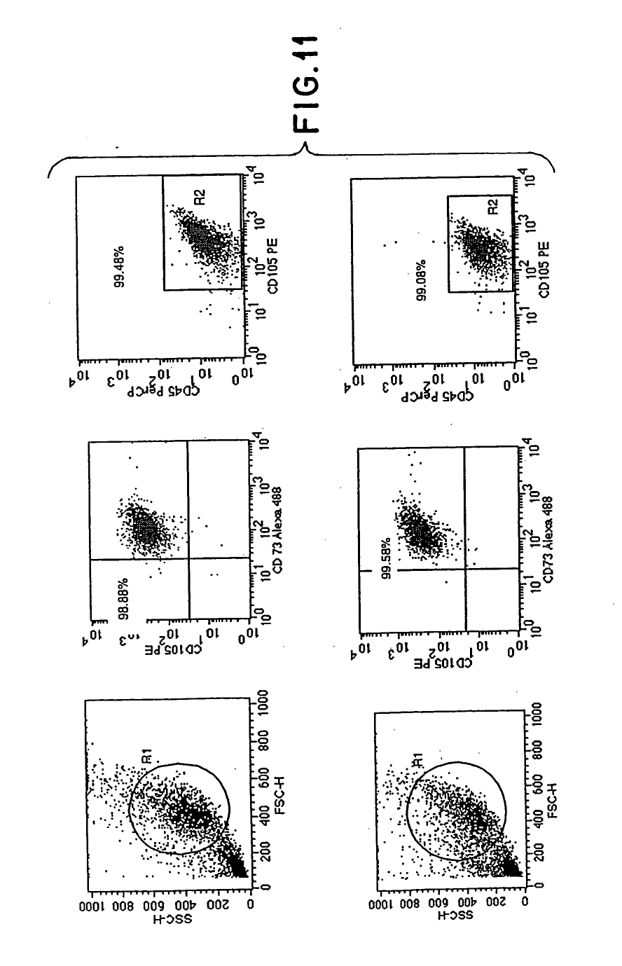

FIG. 12: This Figure shows the Growth kinetics of cord blood MSC cultured in the presence of FBS v/s CBS. Mononuclear cells from cord blood separated on Ficoll-Hypaque were cultured in DMEM: F12 medium for 1 week during which the cells adhered to cell surface. The adhered cells were allowed to become confluent by frequent medium changes. After attaining confluence the cells were trypsinized and cell count taken The trypsinized cells were replated and cultured as mentioned above. At the 3^{rd} passage cells cultured in CBS showed a 3 fold higher cell count as compared to cells cultured in FBS.

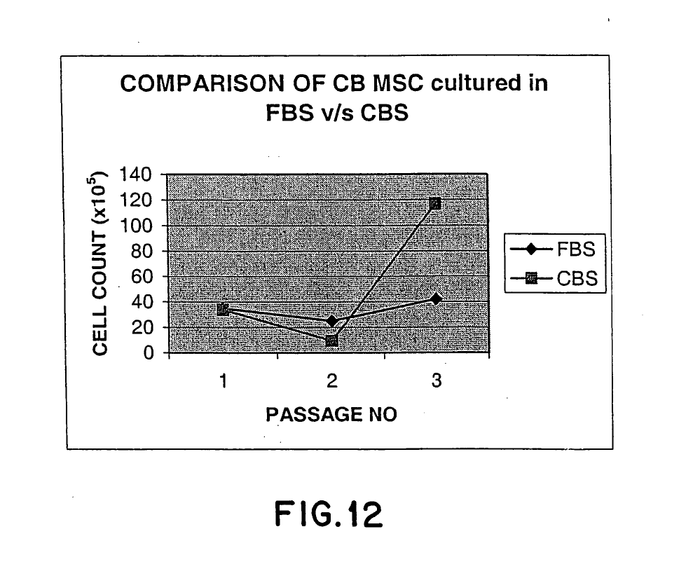

In view of the foregoing descriptions, it will become apparent to those skilled in the art that equivalent modifications thereof may be made without departing from the spirit and scope of this invention.

1. A method of culturing mesenchymal stem calls (hMSC) comprising the steps of culturing these stem cells in a culture medium containing Cord Blood Serum.

2. The method according to claim 1, wherein the cord blood serum is separated from clotted umbilical cord blood for growing mesenchymal stem cells for therapeutic purposes in regenerative medicine.

3. A method as claimed 1, wherein the said culture medium further comprises of
i) a mixture of: Dulbecco's modified Eagle's medium (DMEM) and Ham's F-12 medium and;
ii) β-FGF

4. A method as claimed in claim 2, wherein the Dulbecco's modified Eagle's medium (DMEM) and Ham's F-12 used is in the ratio of 1:1.

5. A method as claimed in claim 1, wherein the Cord Blood Serum used is between 1-50%, preferably between 2-30%.

6. A method as claimed in claim 2, wherein the β-FGF used is 1-50 ng/ml

7. A method as claimed in claim 1, wherein the said Mesenchymal stem cells are obtained from bone marrow.

8. A method as claimed in claim 1, wherein the said Mesenchymal stem cells are obtained from human cord blood.

9. A method as claimed in claim 1, wherein the said Mesenchymal stem cells are obtained from swine bone marrow.

10. A method as claimed in claim 2, wherein the said culture medium expands the said Mesenchymal stem cells.

11. A method as claimed in claim 2, wherein the said medium is complete medium.

12. A method as claimed in claim 1, wherein the said Mesenchymal stem cells are Adult stem cells.

13. A method as claimed in claim 2, wherein the said mesenchymal stem cells may be used for therapeutic purposes in regenerative medicine not limited to cardiac disorders, bone, cartilage and neural disorders.

14. A method of culturing mesenchymal stem calls (hMSC) , substantially as herein described and exemplified in the accompanying examples and in the Figures.
